(19) United States
(12) Patent Application Publication
(10) Pub. No.: US 2024/0050468 A1
(43) Pub. Date: Feb. 15, 2024

(54) FORMULATION COMPRISING ERBIUM BORATE FOR USE IN PREVENTION AND TREATMENT OF WOUND SCARS

(71) Applicant: YEDITEPE UNIVERSITESI, Istanbul (TR)

(72) Inventors: Fikrettin SAHIN, Istanbul (TR); Erhan AYSAN, Istanbul (TR); Pakize Neslihan TASLI, Istanbul (TR); Oguz Kaan KIRBAS, Istanbul (TR); Batuhan Turhan BOZKURT, Istanbul (TR); Berna BULBUL, Balikesir (TR)

(73) Assignee: YEDITEPE UNIVERSITESI, Istanbul (TR)

(21) Appl. No.: 18/268,997

(22) PCT Filed: Nov. 9, 2021
(86) PCT No.: PCT/TR2021/051172

§ 371 Date: Jun. 22, 2023

(30) Foreign Application Priority Data
Dec. 22, 2020 (TR) 2020/21285

(51) Int. Cl. A61K 33/22; A61P 17/02; A61K 47/34; A61K 9/06; A61K 47/32; A61K 47/02; A61K 9/00

(52) U.S. Cl. CPC A61K 33/22; A61P 17/02; A61K 47/34; A61K 9/06; A61K 47/32; A61K 47/02; A61K 9/0014

(57) ABSTRACT

A formulation in gel form comprising erbium borate nanoparticles, at least one surfactant and at least one gelling agent with carrier gel properties for use in prevention and treatment of wound scars is provided.

### CROSS REFERENCE TO RELATED APPLICATIONS

This application is the national phase entry of International Application No. PCT/TR2021/051172, filed on Nov. 9, 2021, which is based upon and claims priority to Turkish Patent Application No. 2020/21285, filed on Dec. 22, 2020, the entire contents of which are incorporated herein by reference.

### FIELD OF THE INVENTION

The present invention relates to a pharmaceutical formulation in gel form comprising erbium borate for use in prevention and treatment of wound scars.

### BACKGROUND

Wounds caused by surgical interventions, traumas, burns and skin infections often cause scarring to be left behind. The said wound scar is called scar in the technical field and is a natural result of the wound healing process. However, scar formation brings along undesirable conditions such as pain and bleeding, as well as being vulnerable to new traumas. The problem that is considered to be the most important about scar formation is that it causes a cosmetically unpleasant appearance. Many pharmaceutical or cosmetic products in the form of creams, ointments and pomades have been developed to prevent scar formation and/or treat the scars that have been formed in the wounds. In addition to the said products, various surgical suture techniques have been tried, but the desired results have not been obtained from the products and the said suture techniques.

The European patent document No. EP0770392, an application in the state of the art, discloses use of salts of various components including erbium salts for obtaining a pharmaceutical, cosmetic or dermatological composition effective against scar formation. In one embodiment of the said invention, the composition of the said invention also includes additional components such as neodymium chloride, glycolic acid, hydroxypropylcellulose, sodium hydroxide, ethanol and preservatives in addition to the erbium salts. The document also discloses that the product of the invention can be in gel form.

In addition to the above, patent applications numbered EP0770392 B1, WO2011/031977 A1, WO2012/065065 A1, EP2988736 A1, EP2983641 and EP0775492 B1 are known as methods of solution in the similar state of art.

### SUMMARY

The objective of the present invention is to provide a pharmaceutical formulation in gel form comprising erbium borate for use in prevention and treatment of wound scars. The gel formulation of the present invention can provide an effective cosmetic result by preventing formation of scars that are probable to be formed after a wound occurs.

### DETAILED DESCRIPTION OF THE INVENTION

The erbium borate-based pharmaceutical formulation of the present invention is used in prevention and treatment of wound scars and comprises erbium borate nanoparticles as active ingredient and at least one surfactant and at least one gelling agent with carrier gel properties.

In one embodiment of the invention, at least one surfactant selected from the group comprising Pluronic F68 (polyoxyethylene-polyoxypropylene block copolymer having an average molecular weight of about 8350 g/mol), Pluronic F127 (polyoxyethylene-polyoxypropylene block copolymer having an average molecular weight of about 12600 g/mol) and mixtures thereof is used as the surfactant.

In one embodiment of the invention, Carbopol (carboxypolymethylene) is used as the gelling agent.

In one embodiment of the invention, the pharmaceutical formulation comprises by mass 0.5-10% Pluronic F68, 0.5-10% Pluronic F127, 0.1-10% Carbopol, 0.5-10% Erbium Borate NP and 0.1-10% Sodium pentaborate.

In a different embodiment of the invention, the pharmaceutical formulation comprises by mass 0.5-10% Pluronic F68, 0.5-10% Pluronic F127, 0.1-10% Carbopol, 0.5-10% Erbium Borate NP, 0.1-10% NaOH and 0.1-10% Chlorhexidine.

The pharmaceutical composition of the present invention can provide an effective cosmetic result by preventing formation of scars that are probable to be formed after a wound occurs. In the in vitro studies carried out during the experimental studies conducted during the development of the invention, it has been observed that in use of the erbium borate-based formulation, expression of the collagen 3 derivative and fibromodulin which are characterized by scarless wound healing increased instead of the collagen 1 derivative which is the main element in wound scar formation. In addition, it was determined that the activity of the matrix metalloprotease protein 9 enzyme, which is very important in prevention of wound scar formation, increased. Furthermore, in an in vivo study conducted on rats, it was found that the erbium borate-based formulation caused less wound scarring when compared to the control group.

What is claimed is:

1. A formulation in gel form for use in prevention and treatment of wound scars, comprising:
erbium borate nanoparticles,
at least one surfactant and
at least one gelling agent with carrier gel properties.

2. The formulation according to claim 1, wherein the at least one surfactant comprises Pluronic F68 and/or Pluronic F127.

3. The formulation according to claim 1, wherein the gelling agent comprises Carbopol.

4. The formulation according to claim 1, wherein the at least one surfactant comprises by mass 0.5-10% Pluronic F68 and 0.5-10% Pluronic F127, the gelling agent comprises 0.1-10% Carbopol, the erbium borate nanoparticles are 0.5-10%, and the formulation further comprising 0.1-10% sodium pentaborate.

5. The formulation according to claim 1, wherein the at least one surfactant comprises by mass 0.5-10% Pluronic F68 and 0.5-10% Pluronic F127, the at least one surfactant comprises 0.1-10% Carbopol, the erbium borate nanoparticles are 0.5-10%, and the formulation further comprises 0.1-10% NaOH and 0.1-10% Chlorhexidine.

6. The formulation according to claim 2, wherein the gelling agent comprises Carbopol.
